(19) United States
(12) Patent Application Publication
(10) Pub. No.: US 2024/0130637 A1
(43) Pub. Date: Apr. 25, 2024

(54) MONITORING VITAL SIGNS OF MULTIPLE PERSONS VIA SINGLE PHASED-MIMO RADAR

(71) Applicant: Rutgers, The State University of New Jersey, New Brunswick, NJ (US)

(72) Inventors: Athina Petropulu, New Brunswick, NJ (US); Yingying Chen, New Brunswick, NJ (US); Zhaoyi Xu, New Brunswick, NJ (US); Chung-Tse Michael Wu, New Brunswick, NJ (US); Cong Shi, New Brunswick, NJ (US); Tianfang Zhang, New Brunswick, NJ (US); Shuping Li, New Brunswick, NJ (US); Yichao Yuan, New Brunswick, NJ (US)

(73) Assignee: Rutgers, The State University of New Jersey, New Brunswick, NJ (US)

(21) Appl. No.: 18/381,416

(22) Filed: Oct. 18, 2023

Related U.S. Application Data
(60) Provisional application No. 63/417,075, filed on Oct. 18, 2022.

(51) Int. Cl. A61B 5/11; A61B 5/05; G01S 7/41; G01S 13/536

(52) U.S. Cl. CPC A61B 5/1126; A61B 5/05; G01S 7/415; G01S 13/536

(57) ABSTRACT

System and method for sensing movement such as chest movement of each of a plurality of target test subjects by: transmitting, at each of N transmitting antennas (TXs) of a phased multiple-input multiple-output (phased-MIMO) radar, a common frequency modulated continuous wave (FMCW) signal in each of a plurality of time division multiplex (TDM) slots, each TDM slot having associated with it a respective weight selected in accordance with a transmit steering vector configured to cause a coherent summation of transmitted signal in a desired direction θ₀ toward at least one target; receiving target-reflected energy associated with the transmitted FMCW signals at a virtual array formed by stacking signal from P TDM slots received via M receiving antennas (RXs) of the phased-MIMO radar; and processing an output of the virtual array to extract therefrom signal received from the desired direction θ₀ to determine thereby target movement in the desired direction θ₀.

### CROSS-REFERENCE TO RELATED APPLICATION

This application claims priority to US Provisional Patent Application No. 63/417,075, filed on Oct. 18, 2022, entitled MONITORING VITAL SIGNS OF MULTIPLE PERSONS VIA SINGLE PHASED-MIMO RADAR (Attorney Docket No. RU-2022-130), which application is incorporated herein by reference in its entirety.

### STATEMENT OF RIGHTS TO INVENTIONS MADE UNDER FEDERALLY SPONSORED RESEARCH

This invention was made with government support under NSF Grants # 2033433 & 1818478. The government has certain rights in the invention.

### FIELD OF THE DISCLOSURE

The present disclosure generally relates motion sensing radar and, in particular, to a time-division multiplexing phased-MIMO radar system.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Vital sign monitoring plays a critical role in tracking the physiological state of people and enabling various health-related applications (e.g., recommending a change of lifestyle, examining the risk of diseases). Traditional approaches rely on hospitalization or body-attached instruments, which are costly and intrusive. However, in recent years there is an emergence of contact-less vital sign monitoring techniques that rely on radio frequency signals. Early studies with continuous wave radars/WiFi devices have shown good success in detecting the vital signs of a single individual, while simultaneous monitoring of the vital signs of multiple, closely spaced subjects remains a challenge.

### SUMMARY

Various deficiencies in the prior art are addressed by systems, methods, architectures, mechanisms or apparatus for sensing movement such as chest movement of each of a plurality of target test subjects by: transmitting, at each of N transmitting antennas (TXs) of a phased multiple-input multiple-output (phased-MIMO) radar, a common frequency modulated continuous wave (FMCW) signal in each of a plurality of time division multiplex (TDM) slots, each TDM slot having associated with it a respective weight selected in accordance with a transmit steering vector configured to cause a coherent summation of transmitted signal in a desired direction θ₀ toward at least one target; receiving target-reflected energy associated with the transmitted FMCW signals at a virtual array formed by stacking signal from P TDM slots received via M receiving antennas (RXs) of the phased-MIMO radar; and processing an output of the virtual array to extract therefrom signal received from the desired direction θ₀ to determine thereby target movement in the desired direction θ₀.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 graphically depicts behavior of a TDM phased-MIMO with 3 TX antennas and 4 RX antennas;

FIG. 2A graphically illustrates DC-offset correction using a least square method, and FIG. 2B graphically illustrates phase drift calibration through interpolation.

FIGS. 3A-3B graphically illustrate respective BR and HR estimation by applying frequency analysis on the phase of mmWave signals;

FIGS. 4A-4B graphically illustrate a comparison of actual (ground truth) and estimated BR and HR, respectively;

FIG. 5 depicts a high-level block diagram of a system according to various embodiments;

FIGS. 6A-6D graphically illustrate a comparison between phased array and phased-MIMO operation for single target BR estimation and corresponding errors;

FIGS. 7A-7D graphically illustrate a comparison between phased array and phased-MIMO operation for single target HR estimation and corresponding errors;

FIG. 8 graphically depicts a main beam formulated by a phased array with 3 TX being unable to entirely cover a target human body at lm from the transmitting radar.

FIGS. 9A-9H graphically illustrate a comparison between phased array and phased-MIMO operation for two targets located at different distances with the same angle separation;

FIGS. 10A-10H, which graphically illustrate a comparison between phased array and phased-MIMO operation for two targets located at the same distance with different angle separation; and

FIGS. 11A-11H graphically illustrate a comparison between phased array and phased-MIMO operation for two targets located at the same distance, same angle separation, but different body orientations.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Various deficiencies in the prior art are addressed by systems, methods, and apparatus providing a time-division multiplexing (TDM) phased-MIMO (multiple-input and multiple-output) radar sensing system allowing high-precision motion sensing for various applications, such as vital sign monitoring of multiple subjects. The various motion sensing / vital sign monitoring embodiments transmit and receive signals with improved range, providing a capability of picking up vital signs at longer distances, for two in while separating the signals of two individuals sitting close together.

### 1 Introduction

Tracking of the physiological states of people can enable change of lifestyle recommendations from indoor sedentary activities and examine the risk of diseases. Vital signs, including breathing rate (BR) and heartbeat rate (HR), provide crucial insights into the physiological state of the individual. Traditional ways to monitor vital signs usually require hospitalization and involve body-attached instruments (e.g., PPG and ECG sensors), which are intrusive, costly, and require the cooperation of the person being monitored. To overcome these problems, research studies have been exploring contact-less vital sign monitoring via radio frequency (RF) signals. Early studies used continuous-wave radar, or WiFi devices transmitting RF signals. The signal echoes are modulated by the small chest movements caused by those vital signs, thus were used for vital sign estimation. However, such methods rely on RF signals that operate at fixed frequencies, and thus have limited ability to disentangle echo signals from targets at different ranges. Such limitation precludes their use in health monitoring of multiple individuals, for example, they could not be used for tracking the vital signs of people in the over-crowded, resource limited clinics that were experienced during the COVID-19 crisis.

Powered by recent advances in mmWave sensing, research studies have been exploring mmWave radars for vital sign monitoring. Compared to low-frequency RF signals, mmWave signals have much shorter wavelength, and thus can better respond to small chest movements, enabling more fine-grained vital sign monitoring. By utilizing frequency-modulated continuous-waveform (FMCW) techniques, a mmWave radar can detect multiple people at different radial distances to the radar device, and further derive vital signs information of each individual.

MmWave FMCW radar for vital sign monitoring have been explored via phased arrays, performing analog beamforming, or multiple-input and multiple-output (MIMO) radar. A phased array approach uses analog beamforming to successively steer the mmWave beam towards different directions. This is achieved by varying the antenna weights so that the transmitted energy is focused in the desired direction. Those works enable the detection of people separated in the angle domain, while the allowable minimum angle separation (resolution) is limited by the receive array's aperture. By processing the echoed signals of each beam separately, the vital signs of the person in each direction can be estimated. For example, some research contemplates deploying analog beamforming on a single-channel FMCW radar to measure the vital signals of two subjects at different ranges with minimum angle separation of 40 degrees. However, the reported preliminary BR and HR estimation performance of a single subject only (i.e., around 93% HR estimation and 96% BR estimation accuracy).

Using a MIMO radar approach allows transmitting mmWave signals via multiple transmitting antennas (TX) in a time-division-multiplexing (TDM) fashion and receiving reflected signal using multiple receiving antennas (RX). By leveraging the TDM-induced orthogonality of the transmitted signals, each receive antenna can extract the contribution of each transmit antenna. The contributions of different TX-RX pairs offer independent views of the targets, which can be exploited to improve target estimation. For example, by using a MIMO radar with 12 TXs and 16 RXs, where in each time slot, only one of the available TXs transmits. The measurements collected over 12 slots correspond to 192 TX-RX antenna pairs and can be used for high-precision vital sign monitoring with low estimation error. All the aforementioned methods consider either analog beamforming, or MIMO techniques, but not both, thus missing the opportunity to fully explore the potential of radar-based mmWave sensing.

Various embodiments provide a novel approach for high-precision vital sign monitoring of multiple people that uses techniques that have not been explored before for vital sign monitoring. In particular, various embodiments provide a TDM phased-MIMO radar sensing scheme that performs transmit and receive beamforming. Various embodiments provide an implementation of the proposed scheme using as basic tool a single-chip automotive mmWave FMCW antenna array and present results for monitoring of the vital signs of two subjects. The unique features of a radar according to the embodiments are as follows:
In each time slot the proposed system transmits a waveform through a phased array structure. The antenna weights in each slot are chosen so that a beam, focusing the transmitted power to the desired direction, is formed. This assumes that the targets have been first detected and their angles are known.
The TDM-MIMO operation of the proposed system enables the formation of a virtual receiving array with aperture longer than that of the physical receiving array. As all transmit-receive pairs corresponding to the virtual array provide independent information about the targets, their combination significantly boosts the receiver signal-to-noise ratio allowing high precision target estimation.
A Capon beamformer (CB) is implemented at the receiver, so that the receiving array focuses on echoes coming from the desired directions, while the power from all other directions in minimized. The large aperture of the virtual receive array enables the receiver to separate closely spaced targets.

Based on the disclosed radar, a system to automatically localize multiple human subjects and estimate their vital signs was developed. The disclosed system first uses Capon's beamformer on the virtual receiving array to obtain the angles of the targets with respect to the radar. Subsequently, the disclosed system iteratively steers the beam towards each individual subject, one subject per slot. The received echoes corresponding to the same subject are processed through a receive Capon beamformer, which focuses on the echoes coming from the desired direction, while minimizing the power from other directions. The narrow beam from the large virtual array aperture enables the separation of the targets, so that each beam contains the vital signs of one subject only.

For each subject, the disclosed system computes the phase of range Discrete Fourier Transform (DFT) peaks corresponding to human subjects, which encodes both the breathing and heartbeats of the subject. Two band-pass filters, which use normal human breathing and heartbeat frequency ranges as cut-off frequencies, are employed to separate the two types of vital signs. The disclosed system then detects the BR and HR in the frequency domain by locating the frequency peaks.

An implementation of the TDM phased-MIMO radar on an off-the-shelf Texas Instrument (TI) AWR2243 mmWave radar with 3 TXs and 4 RXs. The disclosed phased-MIMO radar can steer the mmWave beam towards different directions with a micro-second delay, which enables simultaneously monitoring of the vital signs of multiple individuals. By combining high angle resolution and high precision target estimation, the proposed system enables the precise estimation of the BR and HR of multiple people located close to each other, even when they are at the same radial distance to the radar. Conducting experiments involving two subjects, under various settings (e.g., different distances and angles between the radar and subjects). The results show that the disclosed system can provide high accuracy BR and HR estimation under various experimental settings. The disclosed approach thus provides a promising solution to track the health status of multiple people in many indoor venues (e.g., classrooms, offices, and crowded hospital rooms).

### 2 TDM Phased-MIMO Radar According to Various Embodiments

### 2.1 Analog Transmit Beamforming

A phased-MIMO radar combines MIMO radar and phased array features, in the sense that the radar transmits orthogonal signals, each feeding a phase array structure. Here, orthogonality is achieved by TDM transmission of the same waveform, weighted by different weights in each slot. Orthogonality allows the transmitted signals to be separated at the receiver. The contributions of the multiple orthogonal waveforms offer independent views of the targets, which can be exploited to improve target estimation.

Let us consider a transmitter that has a uniform linear array (ULA) with N TXs spaced by dₜ, and a receiver that has a ULA with M RXs spaced by dᵣ. The transmit array transmits in a time-slotted fashion. In each slot, each TX transmits a weighted version of waveform x(t), using different weights between slots. The weights are chosen so that the transmissions of all antennas add up coherently in a specific direction. By using different weights in each slot, the various embodiments effectively create different channels that provide diversity and thus can lead to improved target estimation.

Let the weights for the p-th slot be:

$$\begin{matrix}{w}_{p} ( θ ) = {{e}^{j 2 π p α ( θ )} [ 1 , {e}^{- j 2 πα ( θ )} , … , {e}^{- j 2 π ( N - 1 ) α ( θ )} ]}^{T} \\ = {e}^{j 2 π p α ( θ )} {a}_{t} ( θ )\end{matrix} where \qquad ( 1 )$$

$$α ( θ ) = {d}_{t} \frac{sin ( θ )}{λ} ,$$

λis the wavelength of signal (transmit wavelength) and aₜ(θ) is a transmit steering vector.

If θ₀ is the direction of the beam, the signal transmitted in the p-th slot towards direction θ can be written as:

$$\begin{matrix}{z}_{p} ( t , θ ) = {a}_{t}^{H} ( θ ) {w}_{p} ( {θ}_{0} ) x ( t ) \\ = {e}^{j 2 π p α ( θ )} {a}_{t}^{H} ( θ ) {a}_{t} ( {θ}_{0} ) x ( t ) ,\end{matrix} \qquad ( 2 )$$

where {·}^{H} denotes the conjugate transpose operation.

The power of transmitted signal at direction θ from the p-th slot equals:

$$\begin{matrix}Q ( θ ) = E \{ {z}_{p} ( t , θ ) {z}_{p}^{H} ( t , θ ) \} \\ = {\vert b ( θ ) \vert}^{2} {Q}_{x}\end{matrix} \qquad ( 3 )$$

where b(θ)=Σₙ₌₀^{N-1}e^{j2 π[α(θ)-α(θ}^{₀}⁾], and Qₓ is the basedband signal power.

One can see that b(θ₀)=N, and the transmitted power is maximized at direction θ₀.

The signal transmitted in each slot is the same as that of a TDM-MIMO radar using the same array, except that it is amplified by the number of antennas.

It is noted that analog beamforming (see Eq. (1)) is not optimal, in the sense that although it makes the signals add up in phase in the preferred direction, it may not control the power in other directions. Optimal transmit beamforming would modulate the power of each TX as well as the phase and can control the sidelobe level. However, optimal transmit beamforming is hard to implement on a phased array.

### 2.2 Virtual Array and Receive Beamforming

At the receiver side, assume that the target is at direction θ₀, after mixing with the conjugate of the transmitted signal, the beat signal in the p-th slot is:

$${y}_{p} ( t ) = {a}_{r} ( {θ}_{0} ) {z}_{p} ( t - τ , {θ}_{0} ) {x}^{H} ( t ) where \qquad ( 4 )$$

$${a}_{r} ( θ ) = [ 1 , {e}^{- j 2 π {d}_{r} \frac{sin θ}{λ}} , … , {e}^{- j 2 π ( M - 1 ) {d}_{r} \frac{sin θ}{λ}} ]$$

is the receive steering vector and τ is the round-trip delay.

By stacking the received signal from P slots, one can formulate a virtual array of PM elements with steering vector:

aᵥ(θ)=aᵣ(θ₀)⊗[1, . . . , e^{j2 π(P-1)α(θ}^{₀}⁾]^{T} (5)

where ⊗ denotes the Kronecker product.

Thus, the virtual array provides a larger aperture than that of the physical receive array. The output of virtual array is:

yᵥ(t)=Naᵥ(θ)x(t−τ)x^{H}(t) (6)

On the outputs of the virtual array, CB is leveraged to focus on the energy of echoed signals coming from a desired direction, while suppressing the energy from other directions. The CB output equals:

z(t)=wᵥHyᵥ(t) (7)

where

$${w}_{v} = \frac{{R}_{y}^{- 1} {a}_{v} ( θ )}{{a}_{v}^{H} ( θ ) {R}_{y}^{- 1} {a}_{v} ( θ )} \qquad ( 8 )$$

with R_{y} being the received signal covariance matrix (estimation details may be found in Section 2.3).

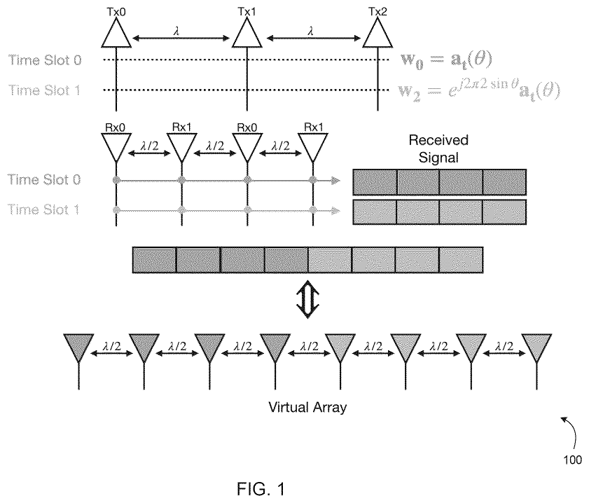

FIG. 1 graphically depicts behavior of a TDM phased-MIMO with 3 TX antennas and 4 RX antennas, to provide thereby a receiving virtual uniform linear array with 8 RX antennas. This provides 8 TX-RX pairs for target estimation. The example of TDM phased-MIMO as shown in FIG. 1 provides a system having 3 TXs spaced apart by a wavelength, and 4 RXs spaced apart by half of wavelength. Two time slots are used to formulate a TDM phased-MIMO with 8 virtual elements. The corresponding 3-dB beam width for such configuration is 18 which supports at maximum 11 non-overlapped beams with distinct directions to monitor vital signals.

By exploiting the multiple channels, corresponding to the multiple TX-RX antenna pairs, phased-MIMO can achieve higher target resolution and higher SNR than a phased array. Also, by implementing transmit beamforming, phased-MIMO can avoid clutter. In vital sign monitoring applications, especially in the multi-target scenario, phased-MIMO can steer the beam towards each of the target and individually monitor their vital signals even when the targets are closely spaced. As shown in Section. 4.4, phased-MIMO can achieve good performance when targets are closely placed and with different orientations.

### 2.3 FMCW Waveforms and Target Parameter Estimation

The proposed radar uses FMCW waveforms, i.e., the transmitted signal is given by (2), where

$$x ( t ) = {A}_{t} {e}^{j 2 π [ {f}_{c} t + \frac{B}{2 {T}_{c}} {t}^{2} + Φ ( t ) ]} . \qquad ( 9 )$$

In the above, Aₜ is the amplitude, f_{c} is the chirp starting frequency, B is the chirp bandwidth, T_{c} is the chirp duration, and Φ(t) is the phase noise from transmitter. Note that the phase noise will be neglected in the following equations since it is slow varying and the propagation delay of mmWave is small. 100481 The transmitted signals in all time slots will be referred to as a frame. Multiple frames are transmitted in sequence to monitor the vital signals of subjects. At the radar receiver, based on (6), the signal received by the m-th RX, due to the transmission of the n-th antenna, i.e., the (nN+m)th element of yᵥ(t), can be written as:

y(n, m, t)=Aₙₘe^{-j2π[f}^{_{b}}^{t+Φ}^{_{b}}^{(t,n,m)]}, (10)

where Aₙₘ is the complex amplitude of the signal transmitted by the n-th transmit antenna and received by the m-th receive antennas after beamforming, and also contains

the effects of the path between the two antennas,

$${f}_{b} = \frac{2 BR ( t )}{{cT}_{c}}$$

is the beat frequency,

$${Φ}_{b} ( t , n , m ) = \frac{2 {f}_{c} R ( t )}{c} - \frac{2 {BR}^{2} ( t )}{{c}^{2} {T}_{c}} - ( {d}_{m} - {d}_{n} ) \frac{sin ( θ )}{λ} ,$$

and R(t) is the radial range of the subject, which is associated and changed with the chest displacements of the target, d=(n−1)dₜ and dₘ=(m−1)dᵣ, respectively.

Since the propagation delay is very small, the phase term can be approximated as

$${Φ}_{b} ( t , n , m ) = \frac{2 {f}_{c} R ( t )}{c} - ( {d}_{m} - {d}_{n} ) \frac{sin ( θ )}{λ}$$

Using discrete-time samples, based on ADC sample interval Tₛ and frame interval T_{f}, the signal of (10) corresponding to the k-th ADC sample of the l-th frame can be written as:

$$y [ n , m , k , l ] = {A}_{nm} {e}^{j 2 π [ {f}_{b} k {T}_{s} + \frac{2 {f}_{c}}{c} R ( {kT}_{s} + {lT}_{f} ) - ( {d}_{m} - {d}_{n} ) \frac{sin ( θ )}{λ} ]} . \qquad ( 11 )$$

Provided that the range change due to vital sign is slow (<2 Hz) and the sampling interval is very short, if the target stays at a nominal range R₀, then the phase term of (11) will be:

$$\begin{matrix}{Φ}_{b} ( l , n , m ) = \frac{2}{λ} [ {R}_{0} + {R}_{1} ( {lT}_{f} ) ] - ( {d}_{m} - {d}_{n} ) \frac{sin ( θ )}{λ} \\ = {Φ}_{0} ( n , m ) + \frac{2 {R}_{1} ( {lT}_{f} )}{λ} .\end{matrix} \qquad ( 12 )$$

where R₁(l) denotes the chest displacement due to vital activities and (11) can be expressed as:

$$y [ n , m , k , l ] = {A}_{nm} {e}^{j 2 {πΦ}_{0} ( n , m )} {e}^{j 2 π {f}_{b} {kT}_{s}} {e}^{j 2 π \frac{2 {R}_{1} ( {lT}_{f} )}{λ}} . \qquad ( 13 )$$

The virtual array may be formulated as follows:

yᵥ(k, l)=[y[0,0, k, l], y[0,1, k, l], . . . y(N−1,M−1, k, l)]^{T}. (14)

To compute the optimal CB weights, an estimate of the covariance matrix based on the available data may be computed, such as follows:

$${\hat{R}}_{y} ( l ) = \frac{1}{K} {\sum }_{k = 0}^{K - 1} {y}_{v} ( k , l ) {y}_{v}^{H} ( k , l ) \qquad ( 15 )$$

Then the covariance estimate is used as the true covariance in (8), and the CB output equals the following:

$$Z [ k , l ] = {w}_{v}^{H} ( l ) {y}_{v} ( k , l ) = \tilde{A} {e}^{j 2 π \frac{2 {R}_{1} ( {lT}_{f} )}{λ}} {e}^{j 2 π {f}_{b} {kT}_{s}} . \qquad ( 16 )$$

$$where \tilde{A} = {\sum }_{n = 0}^{N - 1} {\sum }_{m = 0}^{M - 1} {A}_{nm} .$$

Even if some Aₙₘ are close to zero due to bad channels, Ã will be non-zero. Thus, the subsequent estimation of vital sign signals enjoys high SNR. The disclosed system then leverages the combined signal z[k, l] for BR and HR estimation.

The signal consisting of K samples of z[k, l] during the l-th frame, i.e., z[k, l], k=0, . . . , K−1 can be viewed as a complex sinusoid with frequency f_{b}T_{S} and complex amplitude

$$\tilde{A} {e}^{j 2 π \frac{2 {R}_{1} ( {lT}_{f} )}{λ}} .$$

Therefore, on applying a K-point DFT on z[k, l] along k a peak is seen at DFT sample h=Kf_{b}Tₛ, indicating the radial range of the target. The phase of Ã is constant and can be estimated (see Sec.3.2) by observing the phases of the peak value in all frames. Thus, based on the phases of the DFT sample

$$h = {Kf}_{b} {T}_{s} , i . e . , arg \{ \tilde{A} \} + 2 π \frac{2 {R}_{1} ( {lT}_{f} )}{λ} for l = 0 , ... , L ,$$

one can measure the frequency of chest displacement caused by human chest displacements. As discussed herein, the real and imaginary parts of the peak corresponding to the target in the l-th frame, denoted as Y_{R}[l] and Y_{I}[l], are used.

### 3 Implementation Issues of Vital Sign Monitoring System

TABLE 1
Chirp Parameters used

| | |
|---|---|
| Start Frequency, f_{c}(GHz) | 77 |
| Frequency Slope, S (MHz/μs) | 29.982 |
| Idle Time (μs) | 100 |
| TX Start Time (μs) | 0 |
| ADC Start Time(μs) | 6 |
| ADC Samples | 256 |
| ADC Sample Rate (MHz) | 10 |
| Ramp End Time (μs) | 60 |
| Number of Subframe Per Frame | 2/4 |
| Number of chirp Per Subframe | 1 |
| Slow-time Sampling Frequency, | 20 |
| fₛ = 1/Tₛ (Hz) | |
| Subframe Periodicity (ms) | 12.5 |
| Frame Periodicity (ms) | 50 |

### 3.1 Target Detection

Prior to applying the TDM phased-MIMO technique for multi-people vital sign monitoring, it is useful to detect the human subjects and determine their angles with respect to the radar. This may be accomplished using Capon Beamformer (CB), a widely used angle estimation method.

Upon detecting one or more target subjects, the disclosed system steers the mmWave beam towards the directions of the detected targets by applying analog beamforming at the TX side. The disclosed system then utilizes CB, phased calibration, and frequency analysis techniques as described below to estimate the vital signs of the target subjects.

### 3.2 Constellation Correction with Least-Square

Due to the strong coupling effects and interference in the measurement environment, the range DFT outputs usually contain DC offsets, which distort the phase information at the target's range bin. It is thus useful to remove the DC offsets before deriving reliable vital sign information. Given a TX-RX pair, i, the phase at a selected range bin h can be formulated as

$$ϕ ( l ) = arctan [ \frac{Im ( {r}_{i , h} ( l ) + {DC}_{im} )}{Re ( {r}_{i , h} ( l ) + {DC}_{re} )} ] , \qquad ( 17 )$$

where DCᵢₘ and D Cᵣₑ denote the imaginary and real parts of the complex DC offset, respectively. t denotes the time index, and r_{i,h}(l) shows the range DFT output of the combined signal z[k,l] at range bin h in the l-th frame.

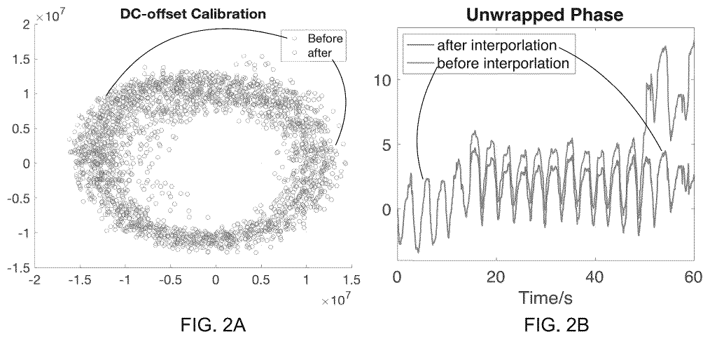

The formulation indicates that the DC offset will shift the origin to (DCᵣₑ, DCᵢₘ). To compensate such a shift, the embodiments may use a least-squares method to estimate and cancel (DCᵣₑ, DCᵢₘ). An example is shown in FIG. 2A, which graphically illustrates DC-offset correction using a least square method, and which shoes that the shifted phases (red dots) are moved so that the center is on the origin (blue dots).

### 3.3 Phase Calculation with DACM

Human chest displacement can exceed the wavelength of mmWave signals (i.e., <4 mm for 77 GHz). Therefore, the phase of the range DFT can be over the range [−π, π], which can lead to false detection of vital signs. To tackle this issue, the embodiments may utilize differential and cross-multiply algorithm (DACM) to calculate phase.

Instead of directly applying arctangent demodulation, DACM converts complex range DFT outputs of l-th frame into phases leveraging the derivative of arctangent function

ϕ(l)=ϕ(l−1)+Δϕ(l), l==2,3, . . . , L, (18)

where L is the number of frames, and

$$Δϕ ( l ) = \frac{{Y}_{R} [ l ] \{ {Y}_{I} [ l ] - {Y}_{I} [ l - 1 ] \} - \{ {Y}_{R} [ l ] - {Y}_{R} [ l - 1 ] \} {Y}_{I} [ l ]}{{{Y}_{R} [ l ]}^{2} + {{Y}_{I} [ l ]}^{2}} .$$

The DACM algorithm mainly corrects the phase distortions caused by breathing. In contrast, small-scale heartbeat motions are less likely to exceed the range of phase.

### 3.4 Phase Drift Calibration Based on Phase Difference

Signal phase drifts in transmission are mainly caused by the impacts of temperature and humidity variations on the hardware, which make the range of phase fluctuations exceed the normal ranges of human breathing and heartbeat. The phase drifts cannot be removed leveraging DACM, since these drifts can be close to but not exceeding the unwrapping threshold of ±π. Furthermore, the harmonics of breathing (i.e., multiple of breathing frequency, e.g., 0.2−0.33 Hz) can also distort the phase patterns at the heartbeat frequency range (e.g., 0.8−2.0 Hz). It is thus necessary to remove the impacts of such harmonic for reliable HR estimation. Particularly, the embodiments may realize phase drift calibration by computing the phase difference Δϕ(l)=ϕ(l)−(l−1) for each ϕ(l) . If the absolute value of the phase difference exceeds a certain threshold, ϕ(l) will be replaced by a new value computed by the Lagrange interpolation using previous three phases ϕ(l−3), ϕ(l−2), ϕ(l−1) and an example is shown in FIG. 2B, which graphically illustrates phase drift calibration through interpolation.

### 3.5 BR and HR Estimation

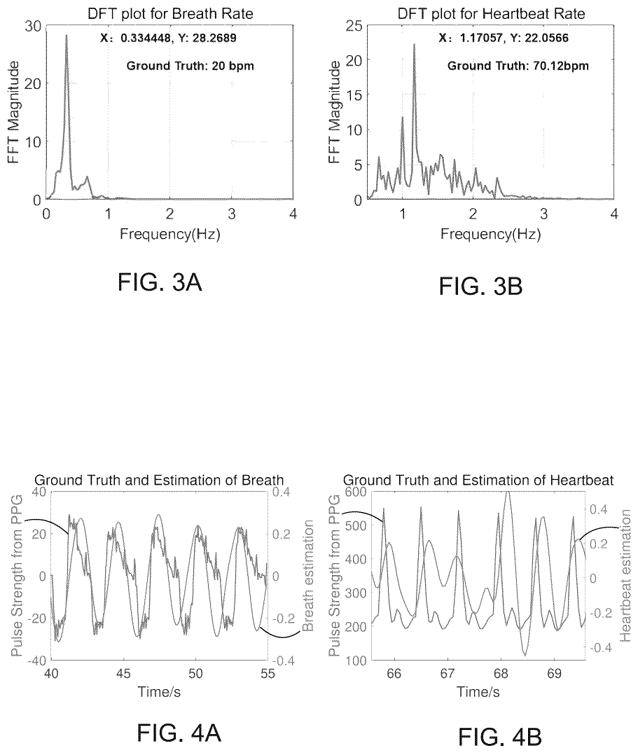

The embodiments may apply frequency analysis upon the calibrated phase within a sliding window to estimate BR and HR. Since the periods of human breathing and heartbeat are close to each other, the embodiments may need to separate the BR and HR for reliable estimations. Particularly, the embodiments may apply a 3rd order Butterworth bandpass filter with a cut-off frequency range of 0.8−2.0 Hz to extract heartbeats, which removes the impacts of human breathing and its harmonics. Similarly, the embodiments may use another bandpass filter of 0.1−0.5 Hz to extract human breaths. Then, the disclosed system applies DFT on the extracted breathing signals, with the highest peak of the DFT magnitude as the detected BR. To extract heartbeat, which involves subtler displacement, the embodiments may first calculate phase difference: Δϕ(l)=ϕ(l)−ϕ(l−1), which reveals minor phase changes. The disclosed system then applies DFT upon the phase differences to calculate the HR. Examples of BR and HR estimation results are shown in FIGS. 3A and 3B, respectively. Comparisons between ground truth and estimation of breath and heartbeat are shown in FIGS. 4A and 4B, respectively.

Performance of the TDM phased-MIMO radar was validated via experiments for multi-target vital sign monitoring conducted using two human subjects seated facing a radar source (front, back, or side facing), at the same or different distances from the radar source (e.g., 166 m), and at the same or different respective angles away from the center transmission of the radar source (e.g., 15° and)30°.

### Illustrative System

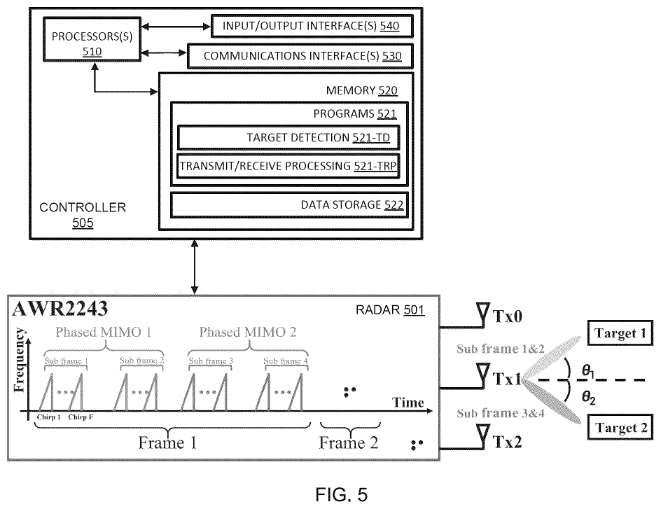

FIG. 5 depicts a high-level block diagram of a system according to various embodiments. The system 500 of FIG. 5 depicts an illustrative embodiment wherein a radar 501 (illustratively a subframe implementation on a TI AWR2243 mmWave device) is configured to transmit via three TX antennas toward a pair of targets as described herein. A controller 505 may be used to control the radar 501 and/or perform various calculations and processing as described herein with respect to the various figures, equations, and disclosure.

As depicted in FIG. 5, the controller 505 includes one or more processors 510, a memory 520, a communications interface 530, and input-output (I/O) interface(s) 540. The processor(s) 510 are coupled to each of memory 520, communication interfaces 530, and I/O interfaces 540.

The processor(s) 510 are configured for controlling the operation of controller 505, including operations supporting the methodologies described herein with respect to the various embodiments. Similarly, the memory 520 is configured for storing information suitable for use by the processor(s) 510. Specifically, memory 520 may store programs 521, data 522 and so on. Within the context of the various embodiments, the programs 521 and data 522 may vary depending upon the specific functions implemented by the controller 505. For example, as depicted in FIG. 5, the programs portion 521 of memory 520 includes a target detection module 521-TD and a transmit/receive processing module 521-TRP (optionally other functional elements/modules) configured to implement various computing, control, management, and/or other functions discussed in this specification.

Generally speaking, the memory 520 may store any information suitable for use by the controller 505 in implementing one or more of the various methodologies or mechanisms described herein. It will be noted that while various functions are associated with specific programs or databases, there is no requirement that such functions be associated in the specific manner. Thus, any implementations achieving the functions of the various embodiments may be used.

The communications interfaces 530 may include one or more services signaling interfaces adapted to facilitate the transfer of information, files, data, messages, requests and the like between various entities in accordance with the embodiments discussed herein.

The I/O interface 540 may be coupled to one or more presentation devices (not shown) such as associated with display devices for presenting information to a user, one or more input devices (not shown) such as touch screen or keypad input devices for enabling user input, and/or interfaces enabling communication between the controller 505 and other computing, networking, presentation or other local or remote input/output devices (not shown).

Various embodiments are implemented using a controller 505 comprising processing resources (e.g., one or more servers, processors and/or virtualized processing elements or compute resources) and non-transitory memory resources (e.g., one or more storage devices, memories and/or virtualized memory elements or storage resources), wherein the processing resources are configured to execute software instructions stored in the non-transitory memory resources to implement thereby the various methods and processes described herein. As such, the various functions depicted and described herein may be implemented at the elements or portions thereof as hardware or a combination of software and hardware, such as by using a general-purpose computer, one or more application specific integrated circuits (ASIC), or any other hardware equivalents or combinations thereof. In various embodiments, computer instructions associated with a function of an element or portion thereof are loaded into a respective memory and executed by a respective processor to implement the respective functions as discussed herein. Thus, various functions, elements and/or modules described herein, or portions thereof, may be implemented as a computer program product wherein computer instructions, when processed by a computing device, adapt the operation of the computing device such that the methods or techniques described herein are invoked or otherwise provided. Instructions for invoking the inventive methods may be stored in tangible and non-transitory computer readable medium such as fixed or removable media or memory or stored within a memory within a computing device operating according to the instructions.

It is contemplated that some of the steps discussed herein as software methods may be implemented within special-purpose hardware, for example, as circuitry that cooperates with the processor to perform various method steps. Portions of the functions/elements described herein may be implemented as a computer program product wherein computer instructions, when processed by a computer, adapt the operation of the computer such that the methods and/or techniques described herein are invoked or otherwise provided. Instructions for invoking the inventive methods may be stored in tangible fixed or removable media, transmitted via a data stream in a broadcast or other tangible signal-bearing medium, and/or stored within a memory within a computing device operating according to the instructions.

Although primarily depicted and described as having specific types and arrangements of components, it will be appreciated that any other suitable types and/or arrangements of components may be used for controller 105.

For example, the controller 505 in whole or in part may be implemented within one or more data centers comprising compute resources (e.g., processing resources such as provided by one or more servers, processors and/or virtualized processing elements or other compute resources), memory resources (e.g., non-transitory memory resources such as one or more storage devices, memories and/or virtualized memory elements or storage resources), input/output (I/0) and communications/network interface resources, and/or other hardware resources and/or combined hardware and software resources suitable for use in implementing a plurality of virtualized machines such as those configured to implement the functions described herein with respect to the controller 505. Various other types of virtualized services platforms, servers, and other known systems may be used to implement the virtualized network management elements such as described herein. The compute or processing resources may also be configured to execute software instructions stored in the non-transitory memory resources to provide thereby the various relay motion control functions, communications path functions, and/or other functions as described herein.

### 4 Performance evaluation

Experimental data generated using an off-the-shelf Texas Instrument automotive FMCW radar confirms that the various embodiments provide a time-division multiplexing (TDM) phased-MIMO radar sensing system that allows high-precision vital sign monitoring of multiple subjects. The various embodiments provide vital sign monitoring in a non-intrusive, contact-free manner (i.e., no cables or sensors attached to a person being monitored), continuous and remote monitoring, as well as simultaneous monitoring of multiple persons.

The proposed sensing system can steer a beam towards desired directions with a micro-second delay. The steerable beam enables capturing the vital signs of multiple individuals at the same radial distance to the radar. The proposed system enables the formation of a virtual array with aperture longer than that of the physical array. A Capon beamformer is used at the receiver side to combine the data collected from different transmit and receive antenna pairs corresponding to the virtual array. As all the pairs provide independent information about the targets, their combination significantly boosts the receiver signal-to-noise ratio. Based on the designed TDM phased-MIMO radar, they developed a system to automatically localize multiple human subjects and estimate their vital signs.

This system can achieve more than 98.06% accuracy for breathing rate (BR) and more than 82.89% accuracy for heartbeat rate (HR) estimation, at a subject-to-radar distance of 1.6 m when the targets are facing the radar. The minimal subject to subject angle separation is 30° at a subject-to-radar distance of 1.6 m, corresponding to a close distance of 0.3 m between two subjects, which outperforms the state-of-the-art.

As discussed herein, evaluations of various embodiments show that under two-subject scenarios, these embodiments are capable of achieving more than 98.06% accuracy for breathing rate (BR) and more than 82.89% accuracy for heartbeat rate (HR) estimation, at a subject-to-radar distance of 1.6 m when the targets are facing the radar.

The minimal subject to-subject angle separation is 30°. at a subject-to-radar distance of 1.6 m, corresponding to a close distance of 0.3 m between two subjects, which outperforms the state-of-the-art. For example, a system according to various embodiments is capable of tracking the breathing rate and heart rate of two people sleeping next to each other. As such, the various embodiments are suitable for use in sleep apnea detection and other surveillance applications where vital sign monitoring without using cables, contact sensors, and the like is desirable.

### 4.1 TDM-Phased-MIMO Implementation

As a proof of concept, an exemplary embodiment of the TDM phased-MIMO radar was implemented using an off-the-shelf TI AWR2243 mmWave device, which transmits and receives FMCW waveforms within 76 GHz˜81 GHz frequency range. The mmWave device consists of three TXs with the spacing of A. and four RXs with the spacing of λ/2 , respectively. It is noted that while the middle TX antenna element is offset from the other two in the elevation direction, the antenna array can still be assumed as a linear antenna array in the horizontal direction given the broadside radiation of the patch antennas and wavefront direction. In the measurement setup, each TX chain includes a programmable phased shifter, with which the analog scanning angle can be achieved by providing different phase information feeding to each TX, separately. The FMCW setting based on AWR2243 was shown in Table 1. In this configuration, the resolution of range bin measurement is 19.5 cm. An evaluation board TI DCA1000 may be adopted in the streaming mode to acquire raw baseband I/Q signals down-converted from received signals.

It is noted that leveraging TXs to realize the analog beamforming, there will be grating lobes pointing to other directions because of the large spacing between TXs, λ>λ/2, which may introduce interference on the multi-target scenario. Nevertheless, RXs are spaced by λ/2 which means there is no grating lobes at the receiver side. By leveraging the CB at the receiver side, the embodiments may alleviate the grating lobe problem since the energy is focused to a certain direction. In multiple-subject scenarios, the disclosed TDM phased-MTh/10 radar can changes the beam direction towards two different subjects within one frame periodicity of 50 ms. Each frame is equally divided into four subframes for multi-target detection, whose block diagram is shown in Fig.5. Particularly, the direction 1 can be illuminated using subframe 1 with TX0 as the reference and subframe 2 with TX2 as the reference, while the direction 2 can be illuminated using subframe 3 with TX0 as the reference and subframe 4 with TX2 as the reference.

TABLE 2
Representative Results of breath rate estimation (single
target scenario and multi-target scenario)

| | | Breath Rate Estimation Results (BPM) | | |
|---|---|---|---|---|
| | | | | PPG |
| | 2*Distance | Phased- | Phased- | (Ground |
| 2*Users | and Angle | Array | MIMO | Truth) |
| User 1 | 1.5 m, 30° | 19.03 | 20.03 | 20.01 |
| User 2 | 3.0 m, −10° | 18.03 | 16.03 | 16.34 |
| User 1(User | 1.5 m(3.0 m), | 18.05 | 16.96 | 17.37 |
| 3) | 30° (−10°) | (19.03) | (20.00) | (20.05) |
| User 3(User | 2.0 m(2.0 m), | 18.67 | 16.56 | 16.12 |
| 4) | 10° (−30°) | (17.02) | (18.03) | (18.14) |

TABLE 3
Representative Results of heartbeat rate estimation
(single target scenario and multi-target scenario)

| | | Heartbeat Rate Estimation Results (BPM) | | |
|---|---|---|---|---|
| | | | | PPG |
| | 2*Distance | Phased- | Phased- | (Ground |
| 2*Users | and Angle | Array | MIMO | Truth) |
| User 1 | 1.5 m, 30° | 62.89 | 84.46 | 84.07 |
| User 2 | 3.0 m, −10° | 61.11 | 61.33 | 61.14 |
| User 1(User | 1.5 m (3.0 m), | 78.22 | 77.62 | 78.06 |
| 3) | 30° (−10°) | (72.10) | (70.81) | (70.83) |
| User 3(User | 2.0 m (2.0 m), | 65.82 | 66.02 | 67.05 |
| 4) | 10 (−30°) | (65.04) | (71.37) | (72.06) |

### 4.2 Experimental Validation and Error Analysis

Evaluation of the performance of the disclosed vital sign monitoring system under single-subject and two-subject scenarios was performed. For both scenarios, experiments were conducted to study the impacts of various factors, including the distance between the radar and the subject and the separation angle between two subjects and the orientations of subjects. A total of 40 experiments was conducted with 2 min time length for each experiment. A total of 6 volunteers participated in the experiments. Breathing and heartbeat signals were collected with a Neulog NUL236 respiration belt and a Neulog NUL208 Heart Rate sensor as the ground truth. A 60-second sliding window, with a step size of 1 second, is applied upon the breathing and heartbeat signals to obtain the ground-truth BR and HR. HR and BR estimated were compared with the disclosed system and the ground truth within the same sliding window for error analysis. To quantify the vital sign estimation performance, statistical metrics including standard derivation (STD), root-mean-square error (RMSE), and estimation accuracy were used. Specifically, STD indicates the consistency of the estimations, and a lower STD means higher consistency and better performance. RMSE measures the average errors between the estimations of the disclosed system and the ground truth. Besides these two statistical metrics, estimation accuracy for evaluation was used, which is defined as the percentage of the estimation with <3 bpm errors.

### 4.3 Single-target Vital Sign Estimation

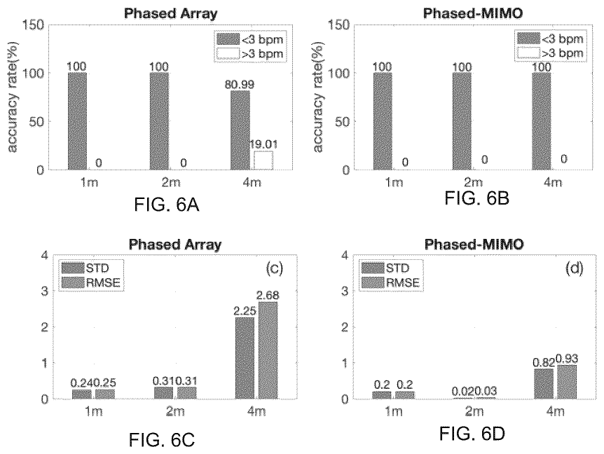

In the scenario of single-target vital sign monitoring, the subject is requested to sit in front of the radar, which is placed 1 m, 2 m and 4 m away from the subject at an angle of 20.1° .The estimation accuracy and statistic results of single-target BR are shown in FIGS. 6A-6D. Examples results of single-target breath and heartbeat estimation are shown in Table 2 and Table 3. For the single-target BR estimation on 1 m and 2 m, the estimation accuracy for phased-array can reach 97%. However, under a far radar-to-subject distance of 4 m, the accuracy drops to 80% for predictions of <3 bpm errors. In contrast, the phased-MIMO not only maintains high accuracy on BR estimation on lm (99%) and 2m (100%), but achieves much higher accuracy at 4 m, with 100% for predictions of <3 bpm errors as well. For the statistic results, the STD and RMSE of both phased-array and phased-MIMO experience a severe increasing when the distance between subjects and radar increases to 4 m, which can attribute to the instability of long-distance data collection from radar. Compared to phased-array, phased-MIMO has much lower STD and RMSE on all three different distances, demonstrating the superior performance on BR estimation.

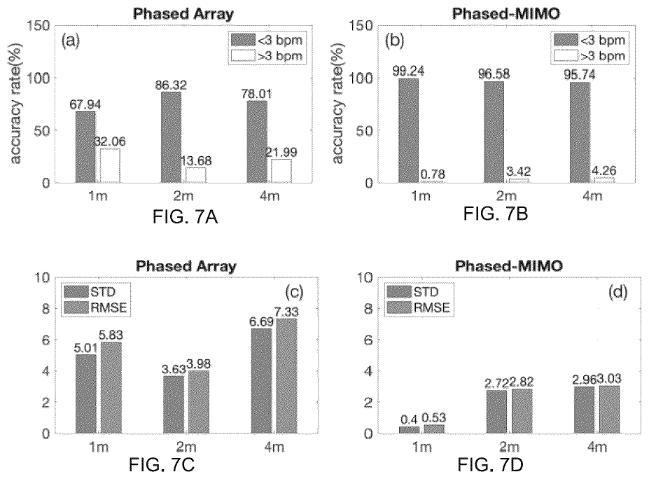

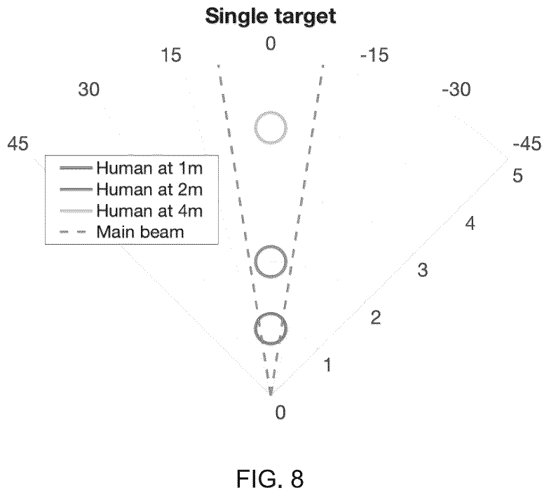

FIGS. 7A-7D graphically illustrate a comparison between phased array and phased-MIMO operation for single target HR estimation and corresponding errors. The x-axis is the target-subject-to-radar distance. Specifically, FIGS. 7A-7D compare the performance of phased array and proposed phased-MIMO on HR estimations. For phased-array, the accuracies are only 67.9%, 86.3%, and 78.0%, on 1 m, 2 m, and 4 m, respectively. In contrast, the proposed phased-MIMO has much better HR estimation performance, with over 95% accuracy of three different distances. It is noted that the phased array has the best performance on the setup of 2 m, with STD of 3.63 and RMSE of 3.98. In order to illustrate this observation, the schematic in FIG. 8 is drawn, where the human body is approximated as a circle with 0.44 m of diameter. As shown in FIG. 8, due to the width of human, when the targets are at 1 m, the beam formulated by the phased array does not cover the whole chest thus leading to a low accuracy. At 2m the beam covers whole chest thus a higher accuracy is achieved. At 4 m, due to attenuation of mmWave signal, the accuracy is lower than in the 2 m case. With the combination of 8 channels, even in longer distance phased-MIMO has lower STD and RMSE, which proves the stability and robustness using phase-MIMO to make single-target heartbeat estimation.

### 4.4 Multi-target Vital Sign Estimation

Multi-target vital signs detection is more challenging, especially when the targets are in the same range bin since a FMCW signal only tells the range information. For that case, analog beamforming and CB were used to separate different targets in angle domain. By transmitting beams towards each of the target, the targets are isolated at the same distance; by CB on the virtual array, a further focus of the energy of received signal towards the desired target is provided, thus reducing the interference from other targets. In the first multi-target detection experiment, two targets are sitting along the direction of −30° and 30° with the same distance of 1 m or 1.6 m away from the radar sensor. In this case, due to the large spacing between TXs, the grating lobe issue appears along the direction of −30° with the maximum gain of main beam along the direction of 30° and vice versa. Fortunately, CB may be used on virtual array to address this problem.

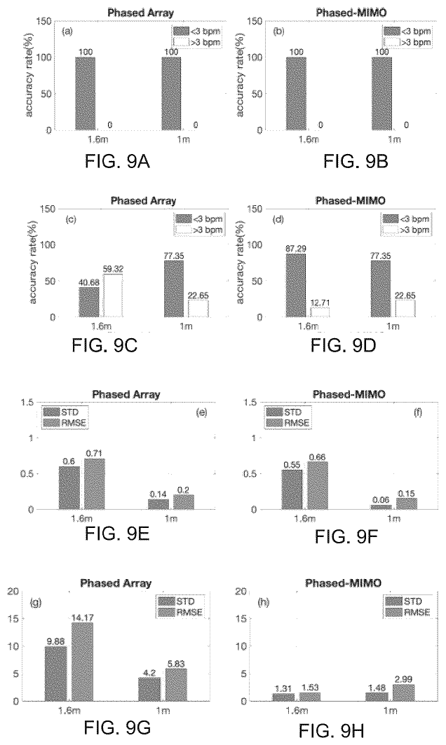

FIGS. 9A-9H graphically illustrate a comparison between phased array and phased-MIMO operation for two targets located at different distances with the same angle separation. Specifically, FIGS. 9A-9B depict accuracy on multi-target BR estimation; FIGS. 9C-9D depict accuracy on multi-target HR estimation; FIGS. 9E-9F depict errors for multi-target BR estimation; and FIGS. 9G-9H depict errors for multi-target HR estimation. The x-axis is subject-to-radar distance, and the angle separation between subjects is 60° .

Example results of multi-target breath and heartbeat estimation are shown in Table 2 and Table 3. The statistic result comparison between phased array and phased-MIMO are shown in FIGS. 9A-9H when two targets located at different distances with the same angle separation of 60°. In FIGS. 9A and 9B, for BR measurement, both the accuracy rates for phased-MIMO and phased array are 100%. At the distance of 1 m, the STD and RMSE for phased array are 0.14 and 0.2, respectively, while the STD and RMSE for phased-MIMO are 0.06 and 0.15, respectively. At the distance of 1.6 m, the STD and RMSE for phased array are 0.6 and 0.71, respectively, while the STD and RMSE for phased-MIMO are 0.55 and 0.66, respectively. For HR measurement in FIGS. 9C and 9D, the accuracy rates between phased array and phased-MIMO are 40.68% and 87.29%, At the distance of 1 m, the STD and RMSE for phased array are 4.2 and 5.83, respectively, while the STD and RMSE of phased-MIMO are 1.48 and 2.99, respectively. At the distance of 1.6 m, the STD and RMSE for phased array are 9.88 and 14.17, respectively, while the STD and RMSE for phased-MIMO are 1.31 and 1.53, respectively.

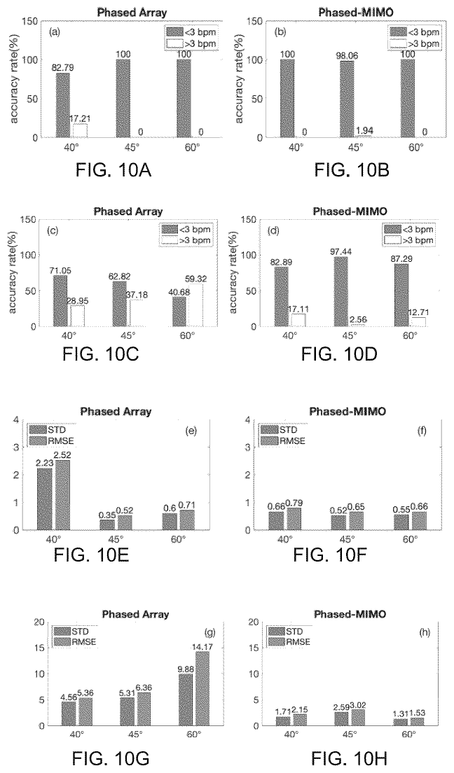

When two targets located at the distance of 1.6m with different angle separations of 40°, 45° and 60°, the performance of phased array and phased-MIMO are summarized in FIGS. 10A-10H, which graphically illustrate a comparison between phased array and phased-MIMO operation for two targets located at the same distance with different angle separation. Specifically, FIGS. 10A-10B depict accuracy on multi-target BR estimation; FIGS. 10C-10D depict accuracy on multi-target HR estimation; FIGS. 10E-10F depict errors for multi-target BR estimation; and FIGS. 10G-10H depict errors for multi-target HR estimation. The x-axis is the angle between two targets, and the radar-to-target distances are all 1.6 m.

In FIGS. 10A and 10B, for BR measurement, the accuracy rate of phased array is larger than 82.79% while the accuracy rate of phased-MIMO is larger than 98.06% when the angle separation is changed from 40° to 60°. For HR measurement shown in FIGS. 10C-10D, the accuracy rate for phased array is larger than 62.82% while the accuracy rate for phased-MIMO is larger than 82.89% when the angle separation is changed from 40° to 60°. Furthermore, FIGS. 10E and 1OF show that the STD and RMSE results for phased-MIMO are smaller than those for phased array. Similar results can be seen in FIGS. 10G-10H. For HR measurement, the accuracy rates for phased-MIMO are larger than those for phased array under different angle separations.

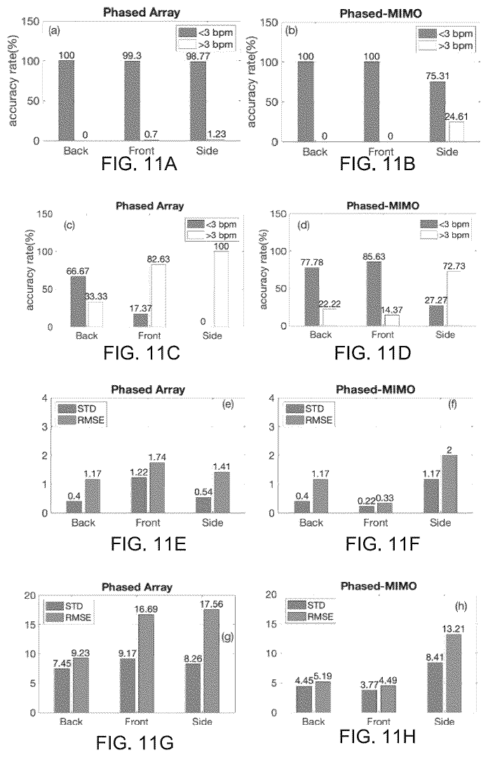

FIGS. 11A-11H graphically illustrate a comparison between phased array and phased-MIMO operation for two targets located at the same distance, same angle separation, but different target/body orientations (i.e., front facing, back facing, and side facing). Specifically, FIGS. 11A-11B depict accuracy on multi-target BR estimation; FIGS. 11C-11D depict accuracy on multi-target HR estimation; FIGS. 11E-11F depict errors for multi-target BR estimation; and FIGS. 11G-11H depict errors for multi-target HR estimation. X-axis: target orientations. The radar-to-target distances are all 1.6 m and inter-target angle is 30°.

FIGS. 11A-11B show the performance of BR measurement employing phased array and phased-MB/10, while FIGS. 11C-11D illustrate the HR measurement performance. FIGS. 11E-11H show STD and RMSE results of phased array and phased-MIMO in this case, respectively. As can be seen in FIGS. 11A-11H, when the radar sensor is used to detect vital sign signals on the front and back chest of target, the accuracy rates for either phased array or phased-MIMO are higher than that when the radar sensor is located on the right side of target. This is mainly because, compared with other two orientations where chest of target is facing to radar, the chest displacement is not obvious on the arm when the radar sensor is located on the right side of target. Furthermore, the reflection area becomes much smaller in that posture, thus the breathing and heartbeat are much more difficult to detect when the radar sensor is located on the right side of target.

### 5 Conclusion

A TDM phased-MIMO radar according to the various embodiments realizes high-precision multi-people vital sign monitoring. The designed radar can successively steer the mmWave beam towards different directions, which enables the separation of vital signals of multiple subjects at the same range bin and integrates the MIMO technique into the embodiments design to boost the SNR. The received echoes are processed with Capon's beamformer to further enhance the directivity which allows us to localize multiple target subjects and extract echoed mmWave signals of each individual subject. As compared to the phased array, the disclosed TDM-phased MIMO with receive beamforming can more accurately estimate the BR and the HR of multiple subjects. Furthermore, compared to the state-of-the-art MIMO-based approach, which relies on a large antenna array (i.e., with 192 TX-RX antenna pairs), the disclosed solution achieves similar performance with the approach on BR detection (0.1 RMSE vs 0.2 RMSE) and HR estimation (0.6 RMSE vs 0.53 RMSE) in single target scenario while using a smaller array of only 8 TX-RX pairs. By implementing the disclosed method on a large antenna array, the performance of vital signal monitoring is may be further improved.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like. It will be appreciated that the term “or” as used herein refers to a non-exclusive “or,” unless otherwise indicated (e.g., use of “or else” or “or in the alternative”).

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A method for sensing movement, comprising:
transmitting, at each of N transmitting antennas (TXs) of a phased multiple-input multiple-output (phased-MTh/10) radar, a common frequency modulated continuous wave (FMCW) signal in each of a plurality of time division multiplex (TDM) slots, each TDM slot having associated with it a respective weight selected in accordance with a transmit steering vector configured to cause a coherent summation of transmitted signal in a desired direction θ₀ toward at least one target;
receiving target-reflected energy associated with the transmitted FMCW signals at a virtual array formed by stacking signal from P TDM slots received via M receiving antennas (RXs) of the phased-MIMO radar; and
processing an output of the virtual array to extract therefrom signal received from the desired direction θ₀ to determine thereby target movement in the desired direction θ₀.

2. The method of claim 1, wherein the desired direction θ₀ comprises the angle of human subjects with respect to the radar and is determined using a Capon Beamformer (CB) angle estimation method.

3. The method of claim 1, wherein each of a plurality of frames are transmitted in sequence toward each of the at least one targets, each transmitted frame comprising a transmitted FMCW signal in each of the plurality of time slots.

4. The method of claim 3, wherein the transmitted FMCW signal is of the form:
$$x ( t ) = {A}_{t} {e}^{j 2 π [ {f}_{c} t + \frac{B}{2 {T}_{c}} {t}^{2} + Φ ( t ) ]}$$
where Aₜ is amplitude, f_{c} is chirp starting frequency, B is chirp bandwidth, Tis chirp duration, and Φ(t) is phase noise from transmitter.

5. The method of claim 3, wherein each m-th RX of the M RXs receives reflected FMCW signal from each n-th TX of the N TXs of the form:
y(n, m, t)=Aₙₘe^{-j2π[f}^{_{b}}^{t+Φ}^{_{b}}^{(t,n,m)]}
where Aₙₘ is the complex amplitude of the signal transmitted by the n-th transmit antenna and received by the m-th receive antennas,
$${f}_{b} = \frac{2 BR ( t )}{{cT}_{c}}$$
is the beat frequency,
$${Φ}_{b} ( t , n , m ) = \frac{2 {f}_{c} R ( t )}{c} - \frac{2 {BR}^{2} ( t )}{{c}^{2} {T}_{c}} - ( {d}_{m} - {d}_{n} ) \frac{sin ( θ )}{λ} ,$$
R(t) is the radial range of the target.

6. The method of claim 1, wherein the phased-MIMO radar transmits via a uniform linear array (ULA) of N transmitting antennas (TXs) spaced by dₜ, and receives via a ULA with M receiving antennas (RXs) spaced by dᵣ.

7. The method of claim 6, wherein for each of the transmitting slot a corresponding weight wₚ(θ) is calculated as:
$${w}_{p} ( θ ) = {e}^{j 2 π p α ( θ )} {a}_{t} ( θ ) ,$$
$$where α ( θ ) = {d}_{t} \frac{sin ( θ )}{λ} ,$$
λ is a transmit wavelength, and aₜ(θ) is a transmit steering vector.

8. The method of claim 1, wherein target movement in the desired direction θ₀ comprises at least one of a heart rate (HR) and a breathing rate (BR) associated with a human target.

9. A vital sign sensing system, comprising:
a phased multiple-input multiple-output (phased-MIMO) radar configured for transmitting, at each of N transmitting antennas (TXs), a common frequency modulated continuous wave (FMCW) signal in each of a plurality of time division multiplex (TDM) slots, each TDM slot having associated with it a respective weight selected in accordance with a transmit steering vector configured to cause a coherent summation of transmitted signal in a desired direction θ₀ toward at least one target;
the phased-MIMO radar configured for receiving, at a virtual array formed by stacking signal from P TDM slots received via M receiving antennas (RXs), target-reflected energy associated with the transmitted FMCW signals; and
processing an output of the virtual array to extract therefrom signal received from the desired direction θ₀ to determine thereby target movement in the desired direction θ₀.

10. The system of claim 9, wherein the desired direction θ₀ comprises the angle of human subjects with respect to the radar and is determined using a Capon Beamformer (CB) angle estimation method.

11. The system of claim 9, wherein each of a plurality of frames are transmitted in sequence toward each of the at least one targets, each transmitted frame comprising a transmitted FMCW signal in each of the plurality of time slots.

12. The system of claim 11, wherein the transmitted FMCW signal is of the form:
$$x ( t ) = {A}_{t} {e}^{j 2 π [ {f}_{c} t + \frac{B}{2 {T}_{c}} {t}^{2} + Φ ( t ) ]}$$
where Aₜ is amplitude, f_{c} is chirp starting frequency, B is chirp bandwidth, T_{c} is chirp duration, and Φ(t) is phase noise from transmitter.

13. The system of claim 11, wherein each m-th RX of the M RXs receives reflected FMCW signal from each n-th TX of the N TXs of the form:
y(n, m, t)=Aₙₘe^{j2π[f}^{_{b}}^{t+Φ}^{_{b}}^{(t,n,m)]}
where Aₙₘ is the complex amplitude of the signal transmitted by the n-th transmit antenna and received by the m-th receive antennas,
$${f}_{b} = \frac{2 BR ( t )}{{cT}_{c}}$$
is the beat frequency,
$${Φ}_{b} ( t , n , m ) = \frac{2 {f}_{c} R ( t )}{c} - \frac{2 {BR}^{2} ( t )}{{c}^{2} {T}_{c}} - ( {d}_{m} - {d}_{n} ) \frac{sin ( θ )}{λ} ,$$
R(t) is the radial range of the target.

14. The system of claim 9, wherein the phased-MIMO radar transmits via a uniform linear array (ULA) of N transmitting antennas (TXs) spaced by dₜ, and receives via a ULA with M receiving antennas (RXs) spaced by dᵣ.

15. The system of claim 14, wherein for each of the transmitting slot a corresponding weight wₚ(θ) is calculated as:
$${w}_{p} ( θ ) = {e}^{j 2 π p α ( θ )} {a}_{t} ( θ ) ,$$
$$where α ( θ ) = {d}_{t} \frac{sin ( θ )}{λ} ,$$
λis a transmit wavelength, and aₜ (θ) is a transmit steering vector.

16. The system of claim 9, wherein target movement in the desired direction θ₀ comprises at least one of a heart rate (HR) and a breathing rate (BR) associated with a human target.

17. A motion sensing system, comprising:
a phased multiple-input multiple-output (phased-MIMO) radar configured for transmitting, at each of N transmitting antennas (TXs), a common frequency modulated continuous wave (FMCW) signal in each of a plurality of time division multiplex (TDM) slots, each TDM slot having associated with it a respective weight selected in accordance with a transmit steering vector configured to cause a coherent summation of transmitted signal in a desired direction θ₀ toward at least one target;
the phased-MIMO radar configured for receiving, at a virtual array formed by stacking signal from P TDM slots received via M receiving antennas (RXs), target-reflected energy associated with the transmitted FMCW signals; and
processing an output of the virtual array to extract therefrom signal received from the desired direction θ₀ to determine thereby target movement in the desired direction θ₀.

18. The system of claim 17, wherein the desired direction desired direction θ₀ comprises the angle of human subjects with respect to the radar and is determined using a Capon Beamformer (CB) angle estimation method.

19. The system of claim 17, wherein each of a plurality of frames are transmitted in sequence toward each of the at least one targets, each transmitted frame comprising a transmitted FMCW signal in each of the plurality of time slots.

20. The system of claim 19, wherein the transmitted FMCW signal is of the form:
$$x ( t ) = {A}_{t} {e}^{j 2 π [ {f}_{c} t + \frac{B}{2 {T}_{c}} {t}^{2} + Φ ( t ) ]}$$
where Aₜ is amplitude, f_{c} is chirp starting frequency, B is chirp bandwidth, T_{c} is chirp duration, and Φ(t) is phase noise from transmitter.
